(19) United States
(12) Patent Application Publication
(10) Pub. No.: US 2026/0315142 A1
(43) Pub. Date: Oct. 15, 2026

(54) SYSTEMS AND METHODS FOR CYLINDRICAL CAGE MAPPING AND ABLATION CATHETERS HAVING INTEGRATED ELECTRODES

(71) Applicant: Biosense Webster (Israel) Ltd., Yokneam (IL)

(72) Inventors: Juan RODRIGUEZ SOTO, Irvine, CA (US); Mohammad ABBAS, Orange, CA (US); Babak EBRAHIMI, Lake Forest, CA (US); Pieter Emmelius VAN NIEKERK, Rancho Santa Margarita, CA (US); Shubhayu BASU, Anaheim, CA (US)

(21) Appl. No.: 19/722,244

(22) Filed: Jun. 26, 2026

Related U.S. Application Data
(63) Continuation of application No. 18/509,900, filed on Nov. 15, 2023, now Pat. No. 12,667,417.
(60) Provisional application No. 63/477,773, filed on Dec. 29, 2022.

(51) Int. Cl. A61B 18/14; A61B 18/16; A61B 18/00; A61B 90/00

(52) U.S. Cl. CPC A61B 18/1492; A61B 18/16; A61B 2018/0016; A61B 2018/00267; A61B 2018/1465; A61B 2018/1467; A61B 2018/1475; A61B 2018/162; A61B 2090/0811

(57) ABSTRACT

The disclosed technology includes basket catheters comprising a generally cylindrical structure having electrodes attached thereto. The disclosed technology can include a structural unit for an end effector comprising a proximal member extending along a longitudinal axis. The proximal member can define a plurality of divergent curvilinear members. Each divergent curvilinear member can define two meander members extending along the longitudinal axis such that the structural unit comprises a plurality of meander members. Each of the plurality of meander members can be connected to a respective convergent curvilinear member of a plurality of convergent curvilinear members. The structural unit can further comprise a distal member connected to the plurality of convergent curvilinear members.

### CROSS REFERENCE TO RELATED APPLICATIONS

This is a continuation of, and claims priority under 35 U.S.C. § 120 to, prior filed U.S. Patent Application No. 18/509,900 filed Nov. 15, 2023 (Attorney Docket No. BIO6768USNP1_253757.434), which claims the benefit of priority under 35 U.S.C. § 119 to prior filed U.S. Provisional Patent Application No. 63/477,773 filed Dec. 29, 2022 (Attorney Docket No. BIO6768USPSP2 - 253757.000318), each of which are hereby incorporated by reference as if set forth in full herein.

### FIELD

The present invention relates generally to medical devices, and in particular to basket catheters comprising a generally cylindrical structure having electrodes attached thereto.

### BACKGROUND

Cardiac arrhythmias, such as atrial fibrillation (AF), occur when regions of cardiac tissue abnormally conduct electrical signals to adjacent tissue. This disrupts the normal cardiac cycle and causes asynchronous rhythm. Certain procedures exist for treating arrhythmia, including surgically disrupting the origin of the signals causing the arrhythmia and disrupting the conducting pathway for such signals. By selectively ablating cardiac tissue by application of energy via a catheter, it is sometimes possible to cease or modify the propagation of unwanted electrical signals from one portion of the heart to another.

Many current ablation approaches in the art utilize radiofrequency (RF) electrical energy to heat tissue. RF ablation can have certain risks related to thermal heating which can lead to tissue charring, burning, steam pop, phrenic nerve palsy, pulmonary vein stenosis, and esophageal fistula.

Cryoablation is an alternative approach to RF ablation that generally reduces thermal risks associated with RF ablation. Maneuvering cryoablation devices and selectively applying cryoablation, however, is generally more challenging compared to RF ablation; therefore cryoablation is not viable in certain anatomical geometries which may be reached by electrical ablation devices.

Some ablation approaches use irreversible electroporation (IRE) to ablate cardiac tissue using nonthermal ablation methods. IRE delivers short pulses of high voltage to tissues and generates an unrecoverable permeabilization of cell membranes. Delivery of IRE energy to tissues using multi-electrode catheters was previously proposed in the patent literature. Examples of systems and devices configured for IRE ablation are disclosed in U.S. Patent Pub. Nos. 2021/0169550A1 (now U.S. Patent No. 11,660,135), 2021/0169567A1, 2021/0169568A1, 2021/0161592A1 (now U.S. Patent No. 11,540,877), 2021/0196372A1, 2021/0177503A1, and 2021/0186604A1 (now U.S. Patent No. 11,707,320), each of which are incorporated herein by reference and attached in the appendix to priority application U.S. 63/477,773.

Basket catheters are commonly used for mapping or ablating cardiac tissue. For example, basket catheters are described in U.S. Pat. Nos. 5,772,590, 6,748,255 and 6,973,340, each of which are incorporated herein by reference and attached in the appendix to priority application U.S. 63/477,773. Basket catheters generally include a plurality of spines attached to the distal end of the catheter and configured to form a generally spherical shape. Each spine typically has at least one electrode attached thereto configured for mapping or ablation of tissue. Manufacturing basket catheters can be a difficult and expensive process due to the small size and complex geometry. In particular, attaching electrodes to the spines can be difficult, time consuming, and expensive. Accordingly, there is a need in the art for improved methods of manufacturing basket catheters.

An example mapping and ablation procedure with a basket catheter can include bringing a basket catheter into contact with tissue (e.g., cardiac tissue) and detecting electrical signals at the tissue to map the electrical signals across the tissue and/or delivering electrical energy to the electrodes to ablate the tissue. Because of the spherical configuration of the basket catheter, spherical basket catheters tend to be well-suited for ablating rounded geometries, such as the pulmonary vein (e.g., for pulmonary vein isolation), but less effective at mapping electrical pulses across planar tissue surface. Therefore, in some instances, multiple catheters must be used to map and/or ablate tissue in a heart. As will be appreciated, using multiple catheters requires the physician to insert and manipulate the multiple catheters, resulting in longer surgical times. Thus, there is a need in the art for catheter designs capable of performing ablation and mapping of planar tissue as well as rounded tissues, such as a pulmonary vein.

### SUMMARY

There is provided in an example of the present disclosure a structural unit for an end effector comprising a proximal member extending along a longitudinal axis. The proximal member can define a plurality of divergent curvilinear members. Each divergent curvilinear member can define two meander members extending along the longitudinal axis such that the structural unit comprises a plurality of meander members. Each of the plurality of meander members can be connected to a respective convergent curvilinear member of a plurality of convergent curvilinear members. The structural unit can further comprise a distal member connected to the plurality of convergent curvilinear members.

The present disclosure includes an end effector for a medical probe. The end effector can comprise a plurality of structural members connected to each other and forming a generally cylindrical structure. Each structural member of the plurality of structural members can comprise a proximal member extending along a longitudinal axis. The proximal member can define a plurality of divergent curvilinear members. Each divergent curvilinear member can define two meander members extending along the longitudinal axis such that the structural member comprises a plurality of meander members. Each of the plurality of meander members can be connected to a respective convergent curvilinear member of a plurality of convergent curvilinear members. The end effector can further include a distal member connected to the plurality of convergent curvilinear members.

The present disclosure includes a structural unit for an end effector comprising a proximal member extending along a longitudinal axis, a first divergent curvilinear member connected to a distal end of the proximal member, a second divergent curvilinear member connected to the distal end of the proximal member, a first meander member connected to a distal end of the first divergent curvilinear member, a second meander member connected to the distal end of the first divergent curvilinear member, a third meander member connected to a distal end of the second divergent curvilinear member, a fourth meander member connected to the distal end of the second divergent curvilinear member, a first convergent curvilinear member, the first meander member and the second meander member being connected to the first convergent curvilinear member, and a second convergent curvilinear member. The third meander member and the fourth meander member can be connected to the second convergent curvilinear member. The structural unit can further comprise a distal member connected to the first convergent curvilinear member and the second convergent curvilinear member.

The present disclosure includes a method of constructing a medical probe. The method can comprise forming a plurality of spines from a planar sheet of material, forming a plurality of electrodes on the planar sheet of material by vacuum deposition, and forming a plurality of electrical traces on the planar sheet of material. Each electrical trace of the plurality of electrical traces can be connected to a respective electrode of the plurality of electrodes. The method can include connecting opposite ends of the planar sheet of material to each other to form a cylindrical structure.

### BRIEF DESCRIPTION OF THE DRAWINGS

The present disclosure will be more fully understood from the following detailed description of the examples thereof, taken together with the drawings in which:

FIG. 1 is a schematic pictorial illustration of a medical system including a medical probe whose distal end includes a basket assembly with electrodes, in accordance with an example of the present invention;

FIG. 2 is a schematic pictorial illustration showing a perspective view of a basket assembly in an expanded form, in accordance with an embodiment of the present invention;

FIG. 3A is a schematic pictorial illustration showing a side view of a basket assembly in an expanded form, in accordance with the disclosed technology;

FIG. 3B is a schematic pictorial illustration showing a side view of a basket assembly in a collapsed form, in accordance with the disclosed technology;

FIG. 4A is a schematic pictorial illustration showing a spine of a basket assembly, in accordance with the disclosed technology;

FIGS. 4B and 4C are schematic pictorial illustrations of a plurality of spines connected together, in accordance with examples of the disclosed technology;

FIG. 5A is a schematic pictorial illustration of a plurality of spines in a collapsed form while FIG. 5B is a schematic pictorial illustration of a plurality of spines in an expanded form, in accordance with the disclosed technology;

FIG. 6A is a schematic pictorial illustration showing a side view of a basket assembly in an expanded form while FIG. 6B is a detail view of an electrode on the basket assembly, in accordance with the disclosed technology; and

FIG. 7 is a flowchart of a method of manufacturing a medical probe, in accordance with the disclosed technology.

### DETAILED DESCRIPTION

As will become apparent throughout this disclosure, the disclosed technology includes several advantages over the prior art. The disclosed technology can include an expandable basket assembly having a generally cylindrical shape. The expandable basket assembly can have a plurality of electrodes attached thereto that can be configured for mapping or ablation of tissue. By including an expandable basket assembly having a generally cylindrical shape, the disclosed technology can be well suited for mapping or ablating generally planar tissue as well as generally circularly-shaped tissue, such as a pulmonary vein. For instance, the generally cylindrical shape can have an approximately planar end having electrodes disposed thereon for mapping or ablating generally planar tissue. The generally cylindrical shape can further include electrodes disposed around an outer circumferential surface that can be configured to mapping or ablating generally circularly-shaped tissue.

The disclosed technology can include electrodes formed directly onto the spines which form the expandable basket assembly. The electrodes can be formed directly onto the spines using vacuum deposition such as physical vapor deposition or chemical vapor deposition. In other words, the process of manufacturing a basket catheter can be simplified because the electrodes are formed directly onto the spine rather than formed separately from the spine and later attached to the spine.

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 110%.

As used herein, the terms “patient,” “host,” “user,” and “subject” refer to any human or animal subject and are not intended to limit the systems or methods to human use, although use of the subject invention in a human patient represents a preferred embodiment. In addition, vasculature of a “patient,” “host,” “user,” and “subject” can be vasculature of a human or any animal. It should be appreciated that an animal can be a variety of any applicable type, including , but not limited thereto, mammal, veterinarian animal, livestock animal or pet type animal, etc. As an example, the animal can be a laboratory animal specifically selected to have certain characteristics similar to a human (e.g., rat, dog, pig, monkey, or the like). It should be appreciated that the subject can be any applicable human patient, for example. As well, the term “proximal” indicates a location closer to the operator or physician whereas “distal” indicates a location further away to the operator or physician.

As discussed herein, “physician” can include a doctor, surgeon, technician, scientist, operator, or any other individual or delivery instrumentation associated with delivery of a multi-electrode catheter for the treatment of drug refractory atrial fibrillation to a subject.

As used herein, the term “meander” refers to a periodic or repeating pattern of structural elements oriented about a center line. A number designating a meander (e.g., a “first meander” or “second meander”) is meant to identify and differentiate two or more different patterns. The two or more different patterns can be the same or similar type of pattern, a mirror image of patterns, or entirely different types of patterns depending on the configuration.

As discussed herein, the term “ablate” or “ablation”, as it relates to the devices and corresponding systems of this disclosure, refers to components and structural features configured to reduce or prevent the generation of erratic cardiac signals in the cells by utilizing thermal energy such as RF ablation or cryoablation, or non-thermal energy, such as irreversible electroporation (IRE), referred throughout this disclosure interchangeably as pulsed electric field (PEF) and pulsed field ablation (PFA). IRE ablation can include monophasic or biphasic pulses. Furthermore, the disclosed technology can include reversible electroporation.

Ablating or ablation as it relates to the devices and corresponding systems of this disclosure is used throughout this disclosure in reference to non-thermal ablation of cardiac tissue for certain conditions including, but not limited to, arrhythmias, atrial flutter ablation, pulmonary vein isolation, supraventricular tachycardia ablation, and ventricular tachycardia ablation. The term “ablate” or “ablation” also includes known methods, devices, and systems to achieve various forms of bodily tissue ablation as understood by a person skilled in the relevant art.

As discussed herein, the terms “tubular" and “tube” are to be construed broadly and are not limited to a structure that is a right cylinder or strictly circumferential in cross-section or of a uniform cross-section throughout its length. For example, the tubular structures are generally illustrated as a substantially right cylindrical structure. However, the tubular structures may have a tapered or curved outer surface without departing from the scope of the present disclosure.

The present disclosure is related to systems, methods or uses and devices which utilize end effectors including cylindrical basket catheters having electrodes directly formed onto the spines. Example systems, methods, and devices of the present disclosure may be particularly suited for mapping and ablation of cardiac tissue to treat cardiac arrhythmias. Ablative procedures incorporating such example catheters can be visualized using fluoroscopy.

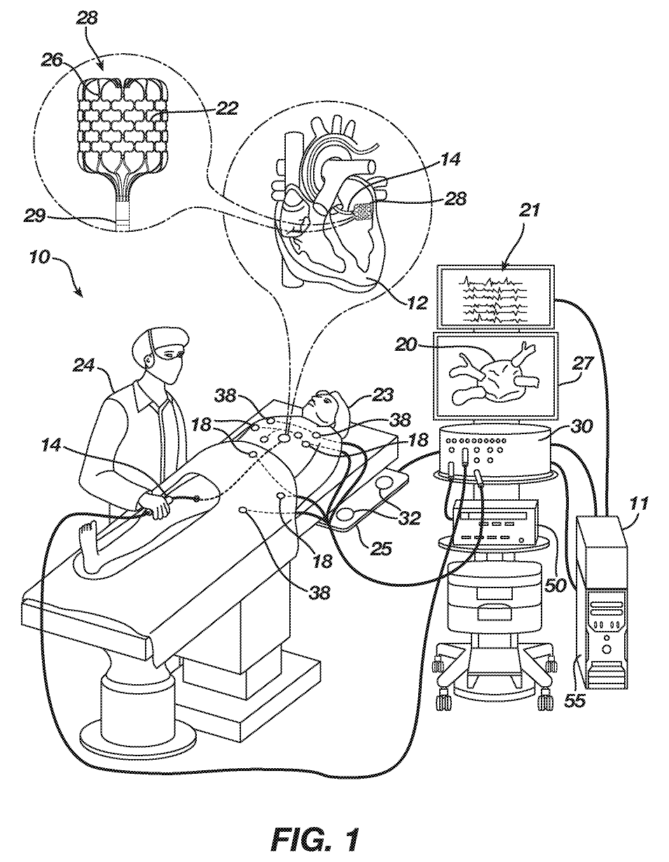

Reference is made to FIG. 1, showing an example catheter-based electrophysiology mapping and ablation system 10. System 10 includes multiple catheters, which are percutaneously inserted by physician 24 through the patient's 23 vascular system into a chamber or vascular structure of a heart 12. Typically, a delivery sheath catheter is inserted into the left or right atrium near a desired location in heart 12. Thereafter, a plurality of catheters can be inserted into the delivery sheath catheter so as to arrive at the desired location. The plurality of catheters may include catheters dedicated for sensing Intracardiac Electrogram (IEGM) signals, catheters dedicated for ablating and/or catheters dedicated for both sensing and ablating. An example catheter 14 that is configured for sensing IEGM is illustrated herein. Physician 24 brings a distal tip (basket assembly 28) of catheter 14 comprising a medical probe 16 into contact with the heart wall at or near the pulmonary vein for sensing a target site in heart 12. For ablation, physician 24 would similarly bring a distal end of an ablation catheter comprising medical probe 16 to a target site for ablating.

Medical probe 16 is an exemplary probe that includes one and preferably multiple electrodes 26 optionally distributed over a plurality of spines 22 at basket assembly 28 and configured to sense the IEGM signals. Medical probe 16 may additionally include a position sensor 29 embedded in or near basket assembly 28 for tracking position and orientation of basket assembly 28. Optionally and preferably, position sensor 29 is a magnetic based position sensor (e.g., an electromagnetic coil) including three magnetic coils for sensing three-dimensional (3D) position and orientation. Position sensor 29 can be conventional coiled wire sensors, flat PCB based sensors, or deformable electromagnetic loop sensors. Although not depicted, position sensor 29 can alternatively be positioned on the basket assembly 28 or designed into individual spines 22. In some embodiments, individual spines 22 can be insulated and act as a position sensor.

In some embodiments, medical probe 16 can include a deformable electromagnetic loop sensor. Examples of various systems and methods for deformable electromagnetic loop sensors are presented in U.S. Patent No.’s 11,304,642 and 10,330,742, and U.S. Patent Publications 2018/0344202A1 and 2020/0155224A1, each of which are incorporated herein by reference and attached in the appendix to priority application U.S. 63/477,773.

Magnetic based position sensor 29 may be operated together with a location pad 25 including a plurality of magnetic coils 32 configured to generate magnetic fields in a predefined working volume. Real time position of basket assembly 28 of catheter 14 may be tracked based on magnetic fields generated with location pad 25 and sensed by magnetic based position sensor 29. Details of the magnetic based position sensing technology are described in U.S. Patent Nos. 5,391,199; 5,443,489; 5,558,091; 6,172,499; 6,239,724; 6,332,089; 6,484,118; 6,618,612; 6,690,963; 6,788,967; 6,892,091, each of which are incorporated herein by reference and attached in the appendix to priority application U.S. 63/477,773.

System 10 includes one or more electrode patches 38 positioned for skin contact on patient 23 to establish location reference for location pad 25 as well as impedance-based tracking of electrodes 26. For impedance-based tracking, electrical current is directed toward electrodes 26 and sensed at electrode skin patches 38 so that the location of each electrode can be triangulated via the electrode patches 38. Details of the impedance-based location tracking technology are described in U.S. Patent Nos. 7,536,218; 7,756,576; 7,848,787; 7,869,865; and 8,456,182, each of which are incorporated herein by reference and attached in the appendix to priority application U.S. 63/477,773.

A recorder 11 displays electrograms 21 captured with body surface ECG electrodes 18 and intracardiac electrograms (IEGM) captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more of electrodes at a distal tip of a catheter configured for ablating. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between catheters, electrophysiological equipment, power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, processor unit with memory or storage with appropriate operating software loaded therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of multiple catheters within the heart chamber, and (4) displaying on display device 27 sites of interest such as places where ablation energy has been applied. One commercial product embodying element of the system 10 is available as the CARTO^{TM}3 System, available from from Biosense Webster, Inc., 31 Technology Drive, Suite 200, Irvine, CA 92618 USA.

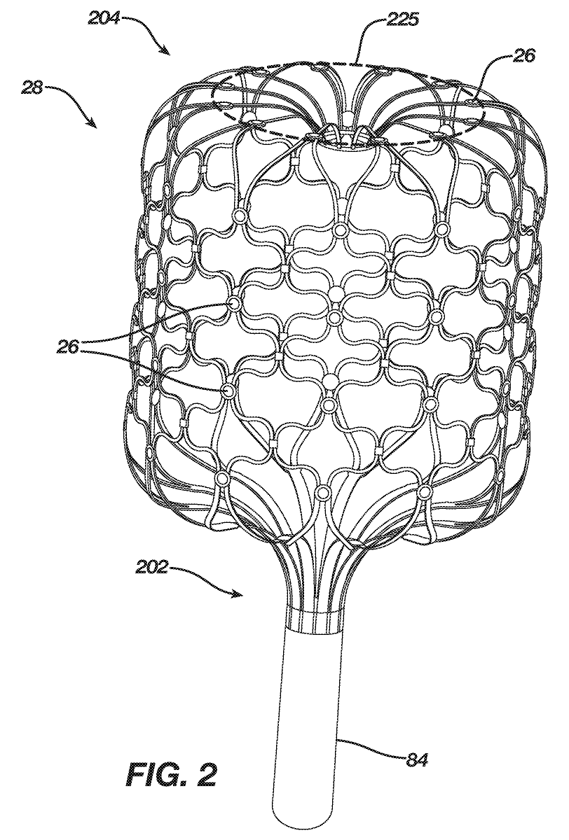

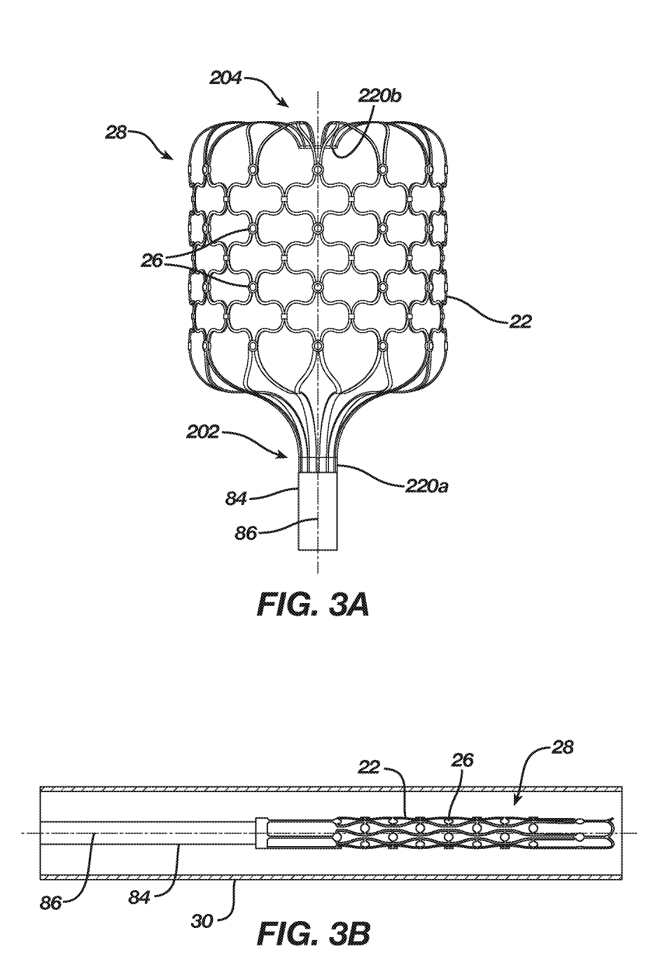

FIG. 2 is a schematic pictorial illustration showing a perspective view of a medical probe 200 in an expanded form while FIG. 3A is a schematic pictorial illustration showing a side view of the medical probe 200 in the expanded form, in accordance with the disclosed technology. The medical probe 200 can be in an expanded form when unconstrained, such as by being advanced out of a delivery sheath 80 by a tubular shaft 84. FIG. 3B, on the other hand, is a schematic pictorial illustration showing a side view of the medical probe 200 in a collapsed form, such as by being retracted into the delivery sheath 80. In the expanded form (FIGS. 2 and 3A), the spines 22 bow radially outwardly along a longitudinal axis 86 and in the collapsed form (FIG. 2B) the spines are constrained generally along the longitudinal axis 86 of tubular shaft 84.

As shown in FIGS. 2 and 3A, the medical probe 200 can include a basket assembly 28 comprising a plurality of flexible spines 22 that are formed at the end of a tubular shaft 84 and are connected at both ends. During a medical procedure, physician 24 can deploy basket assembly 28 by extending tubular shaft 84 from delivery sheath 80 causing the basket assembly 28 to exit the delivery sheath 80 and transition to the expanded form.

As shown in FIGS. 2 and 3A, the basket assembly 28 can include a proximal end 202 and a distal end 204 and form a generally cylindrical shape. The spines 22 can extend between a proximal hub 220a and a distal hub 220b to which the spines 22 can be connected. The distal end 204 can include a generally planar portion 225. As will be described in greater detail herein, the basket assembly 28 can include a plurality of electrodes 26 formed onto the spine 22. Due to the cylindrical shape of the basket assembly 28, the medical probe 200 described herein can be configured for mapping and/or ablation of both planar tissue (e.g., generally planar surfaces of a heart) and rounded tissue locations (e.g., a pulmonary vein). In this way, the medical probe 200 can be configured to perform mapping and/or ablation of multiple surfaces of an organ in a single procedure without requiring the use of multiple catheters having different end effector shapes. The planar portion 225, for example, can have a plurality of electrodes 26 disposed thereon configured for mapping and/or ablation of generally planar tissue and the sides of the cylindrical structure of the basket assembly 28 can include a plurality of electrodes 26 disposed thereon configured for mapping and/or ablation of generally rounded tissue location. The electrodes 26, for example, can be configured to deliver pulses for irreversible electroporation (IRE) with the pulses having a peak voltage of at least 900 volts.

The basket assembly 28 can be formed from a planar sheet of biocompatible material or a cylindrical stock of biocompatible material. For example, the spines 22 can be cut into the biocompatible material and then the basket assembly 28 can be formed into the cylindrical shape by heat setting or otherwise causing the spines 22 to retain the cylindrical shape. The biocompatible material can be any suitable type of biocompatible material including, but not limited to, nitinol, stainless steel, cobalt chromium, polymer material, or other suitable materials.

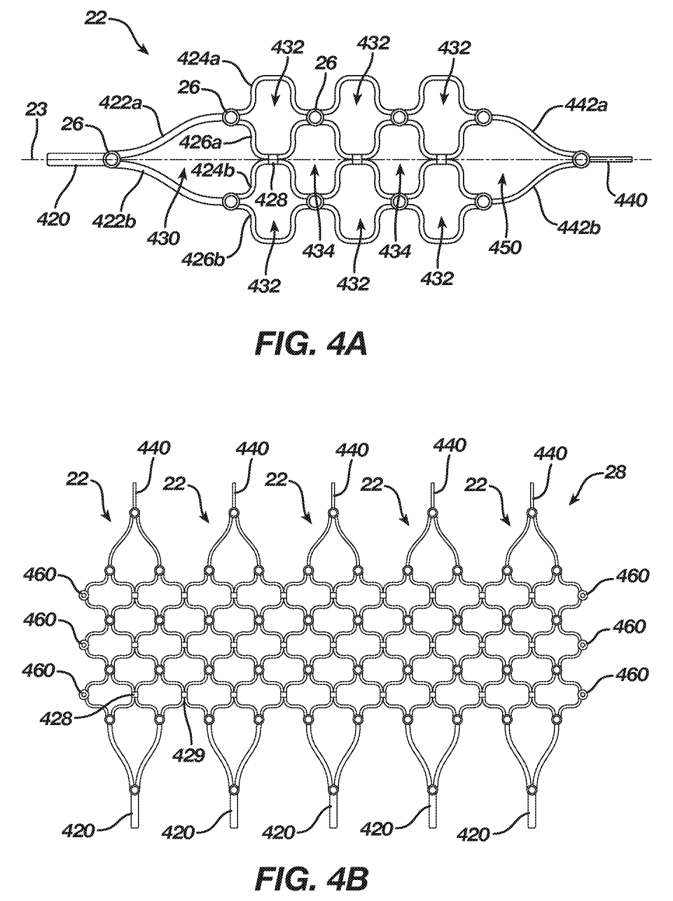

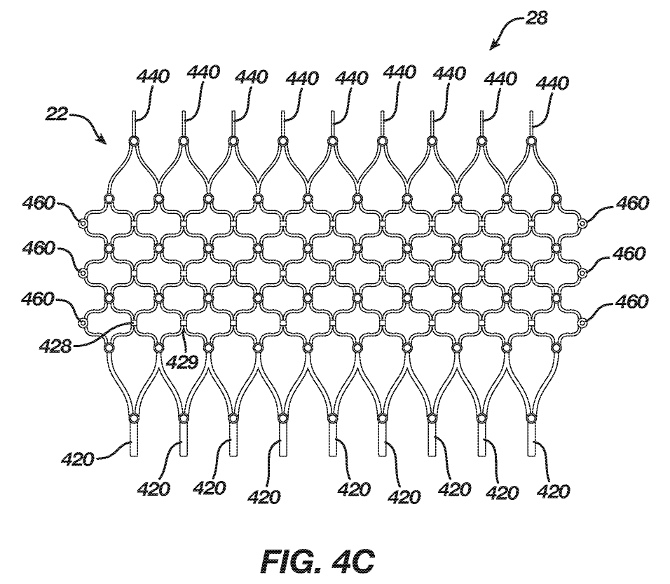

FIG. 4A is a schematic pictorial illustration showing a spine 22 of the medical probe 200, in accordance with the disclosed technology. As will be appreciated, the basket assembly 28 can be made up of several spines 22 connected together. Each spine 22 can have a similar curvilinear shape or pattern as that illustrated in FIG. 4A. Several spines 22 can be connected together to form an array of spines as shown in FIG. 4B. Spines 22 at either end of the array can include connection points 460 which can be connected together to form a generally cylindrical shape. The spine 22 can include a proximal member 420 and a distal member 440. The proximal member 420 can be connected to the proximal hub 220a and the distal member 440 can be connected to the distal hub 420b to form the generally cylindrically-shaped basket assembly 28 shown in FIGS. 2-3B.

As shown in FIG. 4A, and moving from left to right in the figure, the spine 22 can include a proximal member 420 that can be configured for attachment to a proximal hub 220a as just described. The spine 22 can further include a first divergent curvilinear member 422a and a second divergent curvilinear member 422b that can be attached to the proximal member 420 and diverge outwardly from the proximal member 420. Each divergent curvilinear member 422a, 422b can be attached to a first meander member 424a, 424b and a second meander member 426a, 426b that can each form a shape having a repeating pattern as shown in FIG. 4A. The first meander members 424a, 424b and the second meander members 426a, 426b can, for example, form a generally sinusoidal pattern extending generally distally between the respective first divergent curvilinear member 422a and the second divergent curvilinear member 422b and a first convergent curvilinear member 442a and a second convergent curvilinear member 442b. The pattern can be repeated only once or the pattern can be repeated a plurality of times between the respective first and second divergent curvilinear member 422a, 422b and the respective first and second convergent curvilinear member 442a, 442b. For example, the pattern can be repeated once, twice, three times (as shown in FIG. 4A), four times, five times, ten times, twenty times, or any other number of times as would be suitable for the particular configuration. Each meander member 424a, 424b, 426a, 426b can have generally curved turns as shown in FIG. 4A. Alternatively, each meander member 424a, 424b, 426a, 426b can have generally angled or squared turns. The first convergent curvilinear member 442a and the second convergent curvilinear member 442b can each converge toward, and be attached to, a distal member 440.

The spine 22 can be connected at intersections 428 where the second meander member 426a and the first meander member 424b touch as shown in FIG. 4A. In this way, the spine 22 can form a unitary structure and be structurally strengthened. The intersections 428 can be positioned approximately in line with a centerline 23 of the spine 22.

The spine 22 can define various cells at locations between portions of the spine 22. As shown, a proximal cell 430 can be positioned between the first and second divergent curvilinear member 422a, 422b and the second meander member 426a and the first meander member 424b. Similarly, a distal cell 450 can be positioned between the second meander member 426a and the first meander member 424b and the first and second convergent curvilinear members 442a, 442b. Furthermore, intermediary cells 432 can be positioned between first and second meander members 424a, 426a and first and second meander members 424b, 426b.

As shown in FIG. 4A, the spine 22 can have a plurality of electrodes 26 attached thereto at various intersections or points between each member. For example, an electrode 26 can be attached to the spine 22 at a point between the proximal member 420 and the first and second divergent curvilinear member 422a, 422b. Electrodes 26 can be positioned at locations between the respective first and second divergent curvilinear member 422a, 422b and the respective first and second meander members 424a, 424b, 426a, 426b. Electrodes 26 can be positioned between the first and second meander members 424a, 426a and the first and second meander members 424b, 426b. Electrodes 26 can be positioned between respective first and second meander members 424a, 424b, 426a, 426b and respective convergent curvilinear members 442a, 442b. Electrodes can be attached to the spine at a point between the convergent curvilinear members 442a, 442b and the distal member 440.

FIGS. 4B and 4C are schematic pictorial illustrations of a plurality of spines 22 connected together, in accordance with examples of the disclosed technology. The spines 22 can be connected together to form the basket 28. As shown, adjoining spines 22 can be connected together at points 429 wherein adjacent first and second meandering members 424a, 426b touch. As will be appreciated, the spines 22 can be cut from a single sheet of biocompatible material. Therefore, the points 429 can be locations where the sheet of biocompatible material was not cut. In other words, the spines 22 can all be formed together as a continuous material rather than cut individually and then later connected together at the points 420. Alternatively, the spines 22 can be cut individually and then later connected together at the points 420. Furthermore, as will be appreciated, the points 429 and the intersections 428 can be substantially similar except for their position on or between the spine 22.

As shown, spines 22 at either end of the assembled spines 22 can include connection points 460 that can be used to connect the ends of the assembled spines 22 together to form the generally cylindrically shaped basket assembly 28. For example, the spines 22 can all be cut together from a planar sheet of biocompatible material and then the connection points 460 can be aligned with, and connected to, each other to form the basket assembly 28.

As shown in FIG. 4B, the spines 22 can include only a single proximal member 420 and distal member 440 for each individual spine 22 or, as shown in FIG. 4C, the spines 22 can include more than one proximal member 420 and distal member 440 attached to each spine 22. Thus, for the example shown in FIG. 4C, in addition to the points 429, adjoining spines can be connected together via an additional proximal member 420 and distal member 440 and adjoining divergent curvilinear members 422a, 422b and adjoining convergent curvilinear members 442a, 442b.

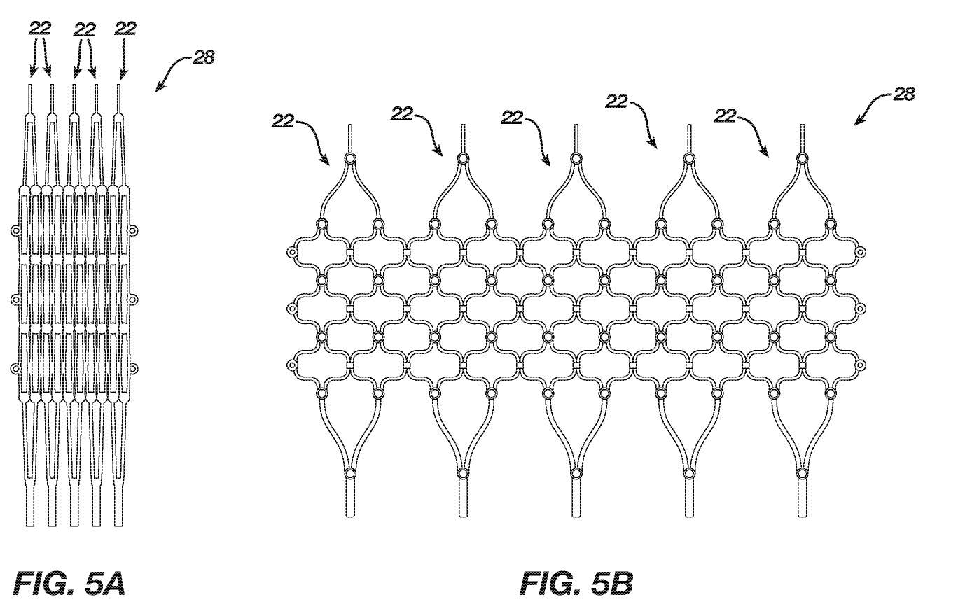

FIG. 5A is a schematic pictorial illustration of a plurality of spines 22 in a collapsed form while FIG. 5B is a schematic pictorial illustration of a plurality of spines 22 in an expanded form, in accordance with the disclosed technology. As described previously, the spines 22 can be cut from a planar sheet of biocompatible material. To reduce material waste, the spines 22 can be cut in the form shown in FIG. 5A and then later stretched or expanded to form the array shown in FIG. 5B.

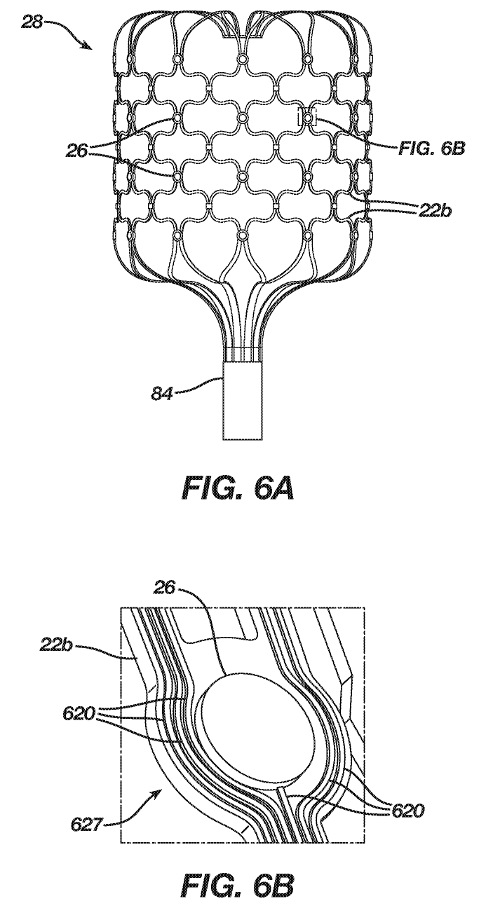

FIG. 6A is a schematic pictorial illustration showing a side view of a basket assembly 28 in an expanded form while FIG. 6B is a detail view of an electrode 26 on the basket assembly 28, in accordance with the disclosed technology. As shown in FIG. 6B, the electrodes 26 can be formed directly onto the spine 22. The electrode 26 can be formed directly onto the spine using, for example, vacuum deposition. The vacuum deposition can include, but is not limited to, physical vapor deposition, chemical vapor deposition, or atomic layer deposition. For vapor deposition, a layer of nitinol can be deposited, followed by a thin insulation layer, and then the conductive trace and electrode layers. As will be appreciated, by forming the electrodes directly onto the spine 22 using vacuum deposition, the disclosed technology can reduce or eliminate many of the complications and errors associated with forming electrodes separate from the spines and then later assembling electrodes onto the spines. In some examples, the electrodes 26 can be formed into the basket assembly by lithography methods, sputtering methods (e.g., spin coating, direct-write sputtering, or sputter coating), printing (e.g., 3D printing), electrodeposition, or photolithography.

The spines 22 can further include electrical leads 620 that can similarly be formed directly onto the spines 22 using vacuum deposition. The electrical leads 620 can be electrically connected to individual electrodes 26 distributed on the spines 22 at various locations. The electrical leads 620 can extend along the length of the spine to the proximal hub 220 where the electrical leads 620 can be electrically connected to wires of the medical probe 17 for sending and/or receiving signals or electrical pulses for mapping and/or ablation.

The basket assembly 28 can include an electrode 26 on a first side of the spine 22 and a reference electrode 627 (on an underside of the spine 22 of FIG. 6B) on a second side of the spine 22. The reference electrode 627 can be configured for receiving far field signals conducted through the blood or other fluid in an organ. As will be appreciated, the far field signals detected by the reference electrode 627 can be received and processed by a computer (e.g., the PIU 30) to compare the signals received from the electrodes 26 to the signals received from the reference electrodes 627 to ensure the signals received for mapping are representative of electrocardiograhic signals through the tissue. Reference electrodes that are configured to measure the electrical signals from the fluid and/or blood directly adjacent to an electrode that is touching tissue. As such, the reference electrodes are insulated from touching tissue and the resulting signals collected are non-local far-field signals. The information from this reference electrode can be used to cancel out far-field signal from the adjacent, tissue touching electrode to ensure that the tissue touching electrode collects local information only.

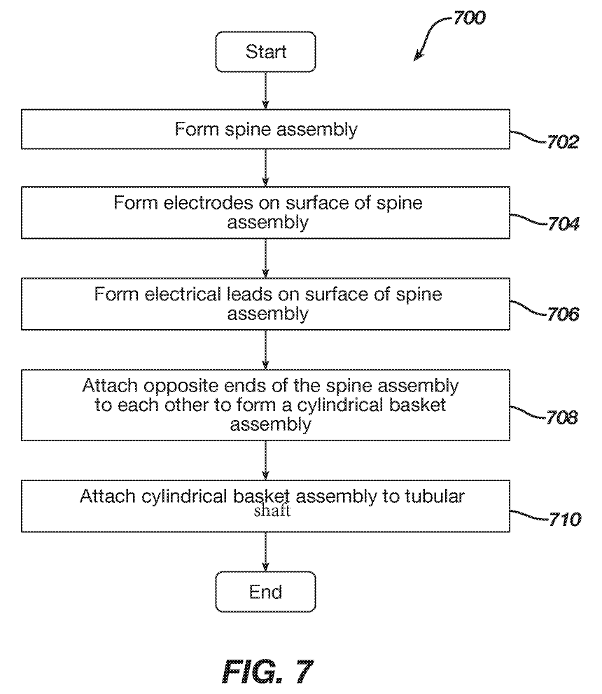

FIG. 7 is a flowchart of a method 700 of manufacturing a medical probe, in accordance with the disclosed technology. The method 700 can include forming a spine assembly from, for example, a planar sheet or cylindrical stock of biocompatible material (step 702). Using, for example, vacuum deposition, the method 700 can include forming electrodes on the surface of the spine assembly and forming 706 electrical leads on the spine assembly (step 704). The method 700 can include attaching opposite ends of the spine assembly to each other to form a cylindrical basket assembly (step 708). The method 700 can further include attaching the cylindrical basket assembly to a tubular shaft to form a generally cylindrically shaped medical probe (step 710). As will be appreciated, the method 700 just described can include any of the previous examples described herein.

The disclosed technology described herein can be further understood according to the following clauses:

Clause 1: A structural unit for an end effector comprising: a proximal member extending along a longitudinal axis, the proximal member defining a plurality of divergent curvilinear members; each divergent curvilinear member defining two meander members extending along the longitudinal axis such that the structural unit comprises a plurality of meander members, each of the plurality of meander members being connected to a respective convergent curvilinear member of a plurality of convergent curvilinear members; and a distal member connected to the plurality of convergent curvilinear members.

Clause 2: The structural unit according to Clause 1, wherein the plurality of divergent curvilinear members comprises two divergent curvilinear members.

Clause 3: The structural unit according to Clause 1 or 2, wherein the plurality of meander members comprises four meander members.

Clause 4: The structural unit according to any of the preceding Clauses, wherein the plurality of convergent curvilinear members comprises two convergent curvilinear members.

Clause 5: The structural unit according to any of the preceding Clauses, wherein each meander member of the plurality of meander members is connected to an adjacent meander member of the plurality of meander members.

Clause 6: The structural unit according to any of the preceding Clauses further comprising a plurality of electrodes attached to the structural unit.

Clause 7: The structural unit according to Clause 6, wherein the plurality of electrodes comprises a first electrode disposed between the proximal member and the plurality of divergent curvilinear members.

Clause 8: The structural unit according to Clauses 6 or 7, wherein the plurality of electrodes comprises one or more electrodes disposed between adjacent meander members of the plurality of meander members.

Clause 9: The structural unit according to Clauses 6-8, wherein each electrode of the plurality of electrodes is formed by vacuum deposition directly onto the structural unit.

Clause 10: The structural unit according to Clause 9, the vacuum deposition comprising physical vapor deposition.

Clause 11: The structural unit according to Clause 9, the vacuum deposition comprising chemical vapor deposition.

Clause 12: The structural unit according to any of Clauses 6-11, further comprising a reference electrode attached to the structural unit.

Clause 13: The structural unit according to Clause 12, wherein the reference electrode is positioned proximate an electrode of the plurality of electrodes.

Clause 14: The structural unit according to any of the preceding Clauses, the structural unit being configured to transition between a collapsed configuration and an expanded configuration.

Clause 15: The structural unit according to any of the preceding Clauses, wherein the structural unit is formed from a planar sheet of resilient material.

Clause 16: The structural unit according to Clause 15, wherein the planar sheet of material comprises nitinol.

Clause 17: An end effector for a medical probe comprising: a plurality of structural members connected to each other and forming a generally cylindrical structure, each structural member of the plurality of structural members comprising: a proximal member extending along a longitudinal axis, the proximal member defining a plurality of divergent curvilinear members; each divergent curvilinear member defining two meander members extending along the longitudinal axis such that the structural member comprises a plurality of meander members, each of the plurality of meander members being connected to a respective convergent curvilinear member of a plurality of convergent curvilinear members; and a distal member connected to the plurality of convergent curvilinear members.

Clause 18: The end effector of Clause 17, wherein the end effector is configured to transition between a collapsed configuration and an expanded configuration.

Clause 19: The end effector according to Clauses 17 or 18, wherein each meander member of the plurality of meander members is connected to an adjacent meander member of the plurality of meander members.

Clause 20: The end effector according to any of Clauses 17-19 further comprising a plurality of electrodes attached to the generally cylindrical structure.

Clause 21: The end effector according to Clause 20, wherein the plurality of electrodes comprises a first electrode disposed between the proximal member and the plurality of divergent curvilinear members.

Clause 22: The end effector according to Clauses 20 or 21, wherein the plurality of electrodes comprises one or more electrodes disposed between adjacent meander members of the plurality of meander members.

Clause 23: The end effector according to any of Clauses 20-22, wherein each electrode of the plurality of electrodes is formed by vacuum deposition directly onto the generally cylindrical structure.

Clause 24: The end effector according to Clause 23, the vacuum deposition comprising physical vapor deposition.

Clause 25: The end effector according to Clause 23, the vacuum deposition comprising chemical vapor deposition.

Clause 26: The end effector according to any of Clauses 20-25, further comprising a reference electrode attached to the generally cylindrical structure.

Clause 27: The end effector according to Clause 26, wherein the reference electrode is positioned proximate an electrode of the plurality of electrodes.

Clause 28: The end effector according to any of the preceding Clauses, wherein the structural unit is formed from a planar sheet of resilient material.

Clause 29: The end effector according to Clause 28, wherein the planar sheet of material comprises nitinol.

Clause 30: The end effector according to any of Clauses 20-29, further comprising one or more electrical traces formed on each structural member, each electrical trace being connected to a respective electrode of the plurality of electrodes.

Clause 31: The end effector according to Clause 30, further comprising a plurality of reference electrodes, each reference electrode of the plurality of reference electrodes being positioned proximate a respective electrode of the plurality of electrodes and attached to a respective structural member of the plurality of structural members on a side of the respective structural member opposite each respective electrode of the plurality of electrodes.

Clause 32: The end effector according to any of Clauses 17-31, further comprising an electromagnetic coil attached to the cylindrical structure, the electromagnetic coil configured to detect a magnetic field for position sensing.

Clause 33: The end effector according to any one of Clauses 17-32, the plurality of structural members being connected to a distal hub at a distal end of the generally cylindrical structure.

Clause 34: The end effector according to Clause 33, wherein the distal hub is positioned proximal to the distal end of the generally cylindrical structure.

Clause 35: A structural unit for an end effector comprising: a proximal member extending along a longitudinal axis; a first divergent curvilinear member connected to a distal end of the proximal member; a second divergent curvilinear member connected to the distal end of the proximal member; a first meander member connected to a distal end of the first divergent curvilinear member; a second meander member connected to the distal end of the first divergent curvilinear member; a third meander member connected to a distal end of the second divergent curvilinear member; a fourth meander member connected to the distal end of the second divergent curvilinear member; a first convergent curvilinear member, the first meander member and the second meander member being connected to the first convergent curvilinear member; a second convergent curvilinear member, the third meander member and the fourth meander member being connected to the second convergent curvilinear member; and a distal member connected to the first convergent curvilinear member and the second convergent curvilinear member.

Clause 36: The structural unit of Clause 35, wherein the first meander member is further connected to the second meander member.

Clause 37: The structural unit of Clauses 35 or 36, wherein the second meander member is further connected to the first meander member and the third meander member.

Clause 38: The structural unit of any of Clauses 35-37, wherein the third meander member is further connected to the second meander member and the fourth meander member.

Clause 39: The structural unit of any of Clauses 35-38, wherein the fourth meander member is further connected to the third meander member.

Clause 40: The structural unit of any of Clauses 35-39 further comprising an electrode connected to the structural unit.

Clause 41: The structural unit of Clause 40, wherein the electrode is positioned between the proximal member, the first divergent curvilinear member, and the second divergent curvilinear member.

Clause 42: The structural unit of Clause 40, wherein the electrode is positioned between the first divergent curvilinear member, the first meander member, and the second meander member.

Clause 43: The structural unit of Clause 40, wherein the electrode is positioned between the second divergent curvilinear member, the third meander member, and the fourth meander member.

Clause 44: The structural unit of Clause 40, wherein the electrode is positioned between the first meander member and the second meander member.

Clause 45: The structural unit of Clause 40, wherein the electrode is positioned between the first meander member, the second meander member, and the first convergent curvilinear member.

Clause 46: The structural unit of Clause 40, wherein the electrode is positioned between the third meander member, the fourth meander member, and the second convergent curvilinear member.

Clause 47: The structural unit of Clause 40, wherein the electrode is positioned between the first convergent curvilinear member, the second convergent curvilinear member, and the distal member.

Clause 48: A method of constructing a medical probe, the method comprising: forming a plurality of spines from a planar sheet of material; forming a plurality of electrodes on the planar sheet of material by vacuum deposition; forming a plurality of electrical traces on the planar sheet of material, each electrical trace of the plurality of electrical traces being connected to a respective electrode of the plurality of electrodes; and connecting opposite ends of the planar sheet of material to each other to form a cylindrical structure.

Clause 49: The method according to Clause 48, wherein the planar sheet of material comprises nitinol.

Clause 50: The method according to Clauses 48 or 49, the cylindrical structure being configured to transition between a collapsed configuration and an expanded configuration.

Clause 51: The medical probe according to any of Clauses 48-50, the cylindrical structure comprising a substantially planar distal portion.

Clause 52: The method according to any of Clauses 48-51, the vacuum deposition comprising physical vapor deposition.

Clause 53: The method according to any of Clauses 48-51, the vacuum deposition comprising chemical vapor deposition.

Clause 54: The method according to any of Clauses 48-53, each spine of the plurality of spines comprising a curvilinear spine.

Clause 55: The method according to any of Clauses 48-54, wherein each electrode is positioned at an intersection between two adjacent spines of the plurality of spines.

Clause 56: The method according to any of Clauses 48-55, further comprising attaching a reference electrode to the cylindrical structure.

Clause 57: The method according to Clause56, further comprising positioning the reference electrode proximate an electrode of the plurality of electrodes.

Clause 58: The method according to Clause 57, further comprising attaching a plurality of reference electrodes to the cylindrical structure, each reference electrode of the plurality of reference electrodes being positioned proximate a respective electrode of the plurality of electrodes and attached to a respective spine of the plurality of spines on a side of the respective spine opposite each respective electrode of the plurality of electrodes.

Clause 59: The method according to any of Clauses 48-58 further comprising attaching an electromagnetic coil to the cylindrical structure, the electromagnetic coil configured to detect a magnetic field for position sensing.

Clause 60: The method according to any one of Clauses 48-59, the plurality of spines converging to a distal hub at a distal end of the cylindrical structure.

Clause 61: The method according to Clause 60, wherein the distal hub is positioned proximal to the distal end of the cylindrical structure.

Clause 62: The method according to any of Clauses 48-61, wherein at least one of the plurality of electrodes is configured for mapping electrical signals in a heart of a patient.

Clause 63: The method according to any of Clauses 48-62, wherein at least one electrode of the plurality of electrodes is configured for ablating tissue in a heart of a patient.

Clause 64: The method according to Clause 63, wherein the at least one electrode is positioned at a distal end of the cylindrical structure.

Clause 65: The method according to Clause 63, wherein the one or more electrodes of the plurality of electrodes are configured to deliver electrical pulses for irreversible electroporation, the pulses having a peak voltage of at least 900 volts (V).

The embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

What is claimed is:

1. A structural unit for an end effector comprising:
a plurality of proximal members extending along a longitudinal axis, the plurality of proximal members defining a plurality of meander members extending axially relative to the longitudinal axis; and
a plurality of distal members connected to the plurality of meander members, and comprising an expanded configuration comprising the plurality of proximal members, the plurality of meander members, and the plurality of distal members forming a generally cylindrical structure comprising:
a circumferential surface portion extending around the longitudinal axis, and
a substantially planar distal surface portion perpendicular to the longitudinal axis.

2. The structural unit according to claim 1, each meander member of the plurality of meander members being connected to an adjacent meander member of the plurality of meander members.

3. The structural unit according to claim 1, further comprising a plurality of electrodes attached to the structural unit.

4. The structural unit according to claim 3, the plurality of electrodes comprising one or more electrodes disposed between adjacent meander members of the plurality of meander members.

5. The structural unit according to claim 1, the structural unit being configured to transition between a collapsed configuration and the expanded configuration.

6. An end effector for a medical probe comprising:
a plurality of structural members connected to each other and forming a generally cylindrical structure, each structural member of the plurality of structural members comprising:
a proximal member extending along a longitudinal axis, the proximal member defining a meander member extending axially relative to the longitudinal axis; and
a distal member connected to the meander member,
the plurality of structural members comprising an expanded configuration forming the generally cylindrical structure comprising a circumferential surface portion extending around the longitudinal axis, and a substantially planar distal surface portion perpendicular to the longitudinal axis.

7. The end effector of claim 6, the end effector being configured to transition between a collapsed configuration and the expanded configuration.

8. The end effector according to claim 6, the meander member of each structural member of the plurality of structural members being connected to an adjacent meander member of the plurality of structural members.

9. The end effector according to claim 6, further comprising a plurality of electrodes attached to the generally cylindrical structure.

10. The end effector according to claim 9, the plurality of electrodes comprising a first electrode disposed between the proximal member and the distal member of each of the plurality of structural members.

11. The end effector according to claim 9, each electrode of the plurality of electrodes being formed by vacuum deposition directly onto the generally cylindrical structure.

12. The end effector according to claim 9, further comprising a reference electrode attached to the generally cylindrical structure.

13. The end effector according to claim 12, the reference electrode being positioned proximate an electrode of the plurality of electrodes.

14. The end effector according to claim 9, further comprising one or more electrical traces formed on each structural member of the plurality of structural members, each electrical trace being connected to a respective electrode of the plurality of electrodes.

15. The end effector according to claim 6, further comprising an electromagnetic coil attached to the generally cylindrical structure, the electromagnetic coil configured to detect a magnetic field for position sensing.

16. An end effector for a medical probe comprising:
a plurality of structural members connected to each other, each structural member of the plurality of structural members defining a meander member extending axially relative to a longitudinal axis, an expanded configuration of the plurality of structural members forming a generally cylindrical structure comprising:
a circumferential surface portion extending around the longitudinal axis;
a planar distal surface portion perpendicular to the longitudinal axis; and
a rounded transition portion connecting the circumferential surface portion to the planar distal surface portion.

17. The end effector according to claim 16, the end effector being configured to transition between a collapsed configuration and the expanded configuration.

18. The end effector according to claim 16, the meander member of each structural member of the plurality of structural members being connected to an adjacent meander member of the plurality of structural members.

19. The end effector according to claim 16, further comprising a plurality of electrodes attached to the generally cylindrical structure.

20. The end effector according to claim 19, further comprising one or more electrical traces formed on each structural member of the plurality of structural members, each electrical trace being connected to a respective electrode of the plurality of electrodes.
